(19) United States
(12) Patent Application Publication
(10) Pub. No.: US 2003/0053971 A1
(43) Pub. Date: Mar. 20, 2003

(54) Sodium hydroxide based hair care compositions comprising ethoxydiglycol

(76) Inventors: John Carson, Union City, NJ (US); Benjamin N. Blinder, Verona, NJ (US)

Correspondence Address: Norris, McLaughlin & Marcus, P.O. Box 1018, Somerville, NJ, 08876-1018, US

(21) Appl. No.: 09/881,362

(22) Filed: Jun. 14, 2001

(51) Int. Cl. A61K 7/06

(52) U.S. Cl. 424/70.1

(57) ABSTRACT

Hair care compositions for relaxing and/or straightening hair having enhanced properties comprising about 1% to about 10% ethoxydiglycol and sodium hydroxide, and methods for making the hair care compositions. The hair care compositions may also comprise emulsifying wax, mineral oil, petrolatum, cetearyl alcohol, water, other active or inert ingredients, colorings and fragrances. The compositions may be used in methods for relaxing and/or straightening hair having reduced processing times.

### BACKGROUND OF THE INVENTION

1. Field of the Invention

The invention relates to hair care compositions comprising ethoxydiglycol. Specifically, the invention relates to hair care compositions for relaxing and/or straightening hair, i.e. hair relaxer/straighteners, comprising from about 1% to about 10% ethoxydiglycol, sodium hydroxide and other ingredients. The hair relaxer/straighteners comprising about 1% to about 10% ethoxydiglycol and sodium hydroxide reduce processing time compared to conventional hair relaxing compositions that do not include ethoxydiglycol.

2. The Prior Art

Ethoxydiglycol, also known as diethylene glycol monoethyl ether, is an ingredient in hair care products. For example, U.S. Pat. No. 2,389,755 describes diethylene glycol monoethyl ether as a stabilizer in a permanent wave formulation; U.S. Pat. No. 4,957,731 describes ethoxydiglycol as an ingredient in a conditioning lotion and U.S. Pat. Nos. 5,700,454; 5,833,966 and 5,989,554 describes diethylene glycol monoethyl ether as an ingredient in a reducing composition.

U.S. Pat. No. 3,912,808 describes compositions for waving or straightening hair comprising high molecular weight water soluble polymers having a multiplicity of amino or quaternary salt groups, with diethylene glycol monoethyl ether as one of many solvents used to solubilize the ingredients of the composition.

U.S. Pat. No. 3,650,280 describes hair thickening products comprising glyoxal, thiourea and/or urea and inert carriers. These hair thickening products may also comprise benzyl alcohol or diethylene glycol monoethyl ether. Hair thickening products are different from hair relaxer/straightener compositions and serve solely to increase the diameter or denier of the hair fiber.

Conventional hair relaxing or straightening compositions generally comprise either lye (sodium hydroxide) as an active ingredient, or guanidine carbonate and calcium hydroxide which upon application react to form the active compound, guanidinium hydroxide. Complete relaxing of the hair, whether using lye or guanidinium hydroxide based compositions, requires about an hour to an hour and a half to complete. Additionally, the treatment can be very painful to the scalp because the treatment compositions, which comprise caustic materials (for example, materials having a pH of about 13.5 and/or sodium hydroxide concentrations over about 2%), can be in direct contact with the scalp for long periods of time (e.g. processing time), for example up to fifteen to twenty minutes. Accordingly, reduced processing time is a desired characteristic for hair relaxer/straighteners in order to reduce the time that the caustic material is in contact with the scalp.

It was an object of the invention to develop hair care compositions, such as hair relaxer/straighteners, which reduce processing time and exposure on the scalp during treatment of the hair and, thus, enhances performance of the hair care compositions.

It was a further object of the invention to develop hair care compositions, such as hair relaxer/straighteners, using components of conventional hair relaxer formulations and ethoxydiglycol.

It was still another object of the invention to develop hair relaxer/straighteners requiring reduced processing time during treatment that does not exhibit decreased or impaired relaxing or straightening capabilities or enhanced scalp discomfort (e.g. irritation or stinging) compared to conventional treatment formulations.

These, and other, objectives are achieved by the hair care compositions described herein, particularly hair relaxer/straighteners, comprising from about 1% to about 10% ethoxydiglycol, sodium hydroxide and other ingredients.

In the present Specification, all parts and percentages are by weight/weight basis unless otherwise specified.

### SUMMARY OF THE INVENTION

The hair care compositions, such as hair relaxer/straighteners, comprise from about 1% to about 10% ethoxydiglycol, sodium hydroxide, emulsifying wax, mineral oil, petrolatum, cetearyl alcohol and water and, optionally, other active or inert ingredients, colorings and/or fragrances. Ethoxydiglycol when used in hair relaxer/straightener compositions comprising sodium hydroxide reduces the processing time compared to conventional hair relaxer/straightener compositions. The compositions also do not increase scalp irritability or decrease performance compared to conventional hair relaxer/straighteners.

The hair care compositions can be made by combining an ethoxydiglycol phase and a sodium hydroxide phase, which comprise the various constituents of the compositions. The hair relaxer/straightener can be applied directly to the hair for up to about 15 minutes to about 20 minutes for treatment.

### DETAILED DESCRIPTION OF THE INVENTION

The hair care compositions comprise from about 1% to about 10% ethoxydiglycol, sodium hydroxide and other components including cetearyl alcohol, petrolatum, mineral oil, emulsifying wax and water. Typically, in addition to the ethoxydiglycol, the hair care compositions will comprise about 1% to about 5% sodium hydroxide solution (30% to 60% solution), about 1% to about 5% cetearyl alcohol, about 10% to about 35% petrolatum, about 5% to about 25% mineral oil, about 5% to about 20% emulsifying wax and about 30% to about 50% water, preferably deionized water. The hair care compositions may further comprise other active or inert ingredients selected from the group consisting of propylene glycol, glycerin, quaternium 80, panthenol, potassium cocoyl hydrolyzed collagen, PEG-60 lanolin, lanolin, steareth-20, laneth-15, PEG-150 stearate, dimethicone, dimethicone copolyol, cyclomethicone, ceteareth-20, PPG-5-ceteth-10 phosphate, polyquaternium-37, trideceth-7, PPG-1-trideceth-6, polysorbate 60, emulsifiers, such as EMULIUM® DELTA available from Gattefossé Corporation (“Gattefossé”), Westwood, N.J., USA and combinations thereof. The compositions may further comprise colorings and/or fragrances.

Ethoxydiglycol is available under the trade name TRANSCUTOL® CG from Gattefossé. Ethoxydiglycol in amounts of about 1% to about 10% of the composition is critical to obtain the beneficial reduced processing time during hair treatment. More enhanced results can be achieved when the amount of ethoxydiglycol is about 3% to about 6%, most preferably about 5%. Amounts greater than about 10% do not yield the beneficial reduced processing time or result in decreased performance compared to the ingredients of sodium hydroxide based conventional hair relaxer/straightener compositions without ethoxydiglycol. The ethoxydiglycol reduces the processing time of the hair relaxer/straightener composition, for example up to about 25%, compared to conventional products, which correlates to reduced processing times of up to about 5 minutes. Ethoxydiglycol was found to be effective in reducing processing time for compositions comprising sodium hydroxide, but the same benefits are not achieved in hair care products where the conventional ingredients comprise guanidine carbonate/calcium hydroxide.

The hair care compositions, i.e. the hair relaxer/straighteners, comprise sodium hydroxide, preferably in a solution comprising about 30% sodium hydroxide to about 60% sodium hydroxide with the balance (about 40% to about 70%) water. This correlates to about 0.5% to about 3% active sodium hydroxide in the hair relaxer/straightener compositions comprising about 1% to about 5% of this sodium hydroxide solution. Addition of ethoxydiglycol to the hair relaxer/straightener compositions in amounts of about 1% to about 10% does not result in negative scalp reaction during hair treatment.

A wide variety of emulsifying waxes may be used in the hair relaxer/straighteners. Preferably, however, nonionic emulsifying waxes are used.

The hair care compositions may be made by combining an ethoxydiglycol phase and a sodium hydroxide phase. The process comprises the following steps which are discusses in greater detail below.

1. Forming an oil phase by combining cetearyl alcohol, petrolatum, mineral oil, emulsifying wax and water, and optionally other active or inert ingredients, coloring and/or flavorings, in a first means for mixing and simultaneously mixing and heating to a temperature of about 70° C. to about 75° C.

2. Forming a water phase by combining ethoxydiglycol and water in a second means for mixing and simultaneously mixing and heating to a temperature of about 70° C. to about 75° C.

3. Combining the oil phase and water phase in the first means for mixing, the second means for mixing or a third means for mixing and simultaneously mixing and cooling to a temperature of about 35° C. to about 40° C. to form an ethoxydiglycol phase.

4. Forming a sodium hydroxide phase by combining water and sodium hydroxide.

5. Combining and mixing the ethoxydiglycol phase and sodium hydroxide phase to obtain the hair care composition.

It is understood that the processing may comprise additional steps or procedures as described herein.

The ethoxydiglycol phase is made by combining an oil phase and a water phase. The oil phase is made by combining sufficient amounts of cetearyl alcohol, petrolatum, mineral oil, emulsifying wax, and optionally, other active or inert ingredients, colorings and/or fragrances in a first means for mixing such as a vessel equipped with at least one stirrer, propeller and/or impeller, and simultaneously mixing and heating the contents to a temperature of about 70° C. to about 75° C. The water phase is made by combining sufficient amounts of ethoxydiglycol and water, preferably deionized water, in a second means for mixing such as a vessel equipped with at least one stirrer, propeller and/or impeller and simultaneously mixing and heating the contents to a temperature of about 70° C. to about 75° C. The oil phase and water phase are combined and simultaneously mixed and cooled to about 35° C. to about 40° C. in the first means for mixing, second means for mixing or a third means for mixing, such as those devices discussed above with respect to the first and second means for mixing, or a colloid mill or a mixer emulsifier, to obtain the ethoxydiglycol phase which may be in the form of an emulsion.

The sodium hydroxide phase comprising water, preferably deionized water, and a sufficient amount of sodium hydroxide to obtain a final product hair care composition having from about 0.5% to about 3% active sodium hydroxide is formed in a mixer and then added to the ethoxydiglycol phase and the ethoxydiglycol phase and sodium hydroxide phase are mixed together at ambient conditions to form the hair care composition without further processing. Optionally, after standing for up to about 24 hours at ambient conditions the mixture of the ethoxydiglycol phase and sodium hydroxide phase can be cooled to about 35° C. to about 40° C. and then homogenized by techniques that would be understood by one skilled in the art. The hair care composition will have a viscosity of about 100,000 centipoise to about 300,000 centipoise, with higher viscosities achieved by homogenization.

In a variation of the process, the ethoxydiglycol phase can be allowed to stand at room temperature for up to about 24 hours prior to mixing in the sodium hydroxide phase at ambient temperatures, in which event the mixture is immediately cooled to about 35° C. to about 40° C. and homogenized to obtain the hair care composition. Sufficient amounts of ingredients in the process means amounts of separate ingredients to obtain a final composition comprising about 1% to about 10% ethoxydiglycol, about 0.5% to about 3% active sodium hydroxide, about 1% to about 5% cetearyl alcohol, about 10% to about 35% petrolatum, about 5% to about 25% mineral oil, about 5% to about 20% emulsifying wax and about 30% to about 50% deionized water.

The hair care compositions are used for straightening and/or relaxing hair. A method for relaxing and straightening hair using the hair care compositions comprises applying the hair care composition to human hair, preferably in stiff cream form, and allowing the hair care composition to remain in contact with the hair for up to about 15 minutes, preferably up to about 20 minutes, then rinsing completely.

### EXAMPLES

### Example 1

Sufficient ethoxydiglycol, TRANSCUTOL CG from Gattefossé, was added to REVLON REALISTIC® EXTRA CONDITIONING™ Cremé Relaxer to make test formulations comprising 5%, 10% and 20% ethoxydiglycol. About 10 grams of each test formulation was applied to separate tresses of hair weighing about 6 grams each. Additionally, a control formulation of about 10 grams of the REVLON REALISTIC EXTRA CONDITIONING Cremé Relaxer, without ethoxydiglycol, was applied to separate hair tresses weighing about 6 grams each for comparison purposes.

The hair tresses were observed for relaxing and straightening effects for up to about 24 minutes after initial application. Observations were noted for no relaxation, slight relaxation, some relaxation and good relaxation (with the quantity of relaxation increasing incrementally from no relaxation to good relaxation) at the time intervals noted in Table 1. The observations are set forth in Table 1 below.

TABLE 1

| Time | | | 10% | 20% |
|---|---|---|---|---|
| Min- | 0% | 5% | TRANS- | TRANS- |
| utes | TRANSCUTOL | TRANSCUTOL | CUTOL | CUTOL |
| 5 | no relaxation | no relaxation | no | no |
| | | | relaxation | relaxation |
| 10 | no relaxation | slight relaxation | no | slight |
| | | | relaxation | relaxation |
| 12 | slight relaxation | slight relaxation | slight | slight |
| | | | relaxation | relaxation |
| 14 | slight relaxation | slight relaxation | no | no |
| | | | relaxation | relaxation |
| 16 | slight relaxation | no relaxation | no | slight |
| | | | relaxation | relaxation |
| 18 | slight relaxation | slight relaxation | slight | some |
| | | | relaxation | relaxation |
| 20 | slight relaxation | slight relaxation | some | some |
| | | | relaxation | relaxation |
| 22 | slight to some | slight to some | some | good |
| | relaxation | relaxation | relaxation | relaxation |
| 24 | good relaxation | good relaxation | good | good |
| | | | relaxation | relaxation |

### Example 2

The test and control formulations of Example 1 were applied to additional hair tresses in the manner set forth in Example 1. The hair tresses were observed for up to 21 minutes for relaxation and straightening and the observations for relaxation of the hair at various time increments after application are set forth in Table 2.

TABLE 2

| Time | | | 10% | 20% |
|---|---|---|---|---|
| Min- | 0% | 5% | TRANS- | TRANS- |
| utes | TRANSCUTOL | TRANSCUTOL | CUTOL | CUTOL |
| 5 | no relaxation | no relaxation | no relaxation | no relaxation |
| 10 | no relaxation | no relaxation | no relaxation | no relaxation |
| 15 | no relaxation | good relaxation | no relaxation | some |
| | | | | relaxation |
| 17 | some | good relaxation | some | good |
| | relaxation | | relaxation | relaxation |
| 19 | good relaxation | good relaxation | good | good |
| | | | relaxation | relaxation* |
| 21 | good relaxation | good relaxation | good | good |
| | | | relaxation | relaxation* |

*These are not as elastic as the other samples - they may not be as completely relaxed.

### Example 3 (Comparative)

Test formulations were made by adding sufficient ethoxydiglycol, TRANSCUTOL CG from Gattefossé, to DARK & LOVELY™ Regular Ultra-Deep Conditioning No-Lye Relaxer available from Carson Products Co., Savannah, Ga., USA, to make test formulations comprising 5%, 10% and 20% ethoxydiglycol. DARK & LOVELY conditioner comprises a guanidine carbonate/calcium hydroxide reactive system. About ten grams of each test formulation and a control formulation of DARK & LOVELY conditioner without ethoxydiglycol were applied to separate hair tresses weighing about 6 grams each as in Example 1.

The tresses were observed for relaxing and straightening effects for up to about 20 minutes after application or until straightening was achieved. The observations at various time increments after application are set forth in Table 3.

TABLE 3

| Time | 0% | 5% | | |
|---|---|---|---|---|
| Min- | TRANS- | TRANS- | 10% | 20% |
| utes | CUTOL | CUTOL | TRANSCUTOL | TRANSCUTOL |
| 5 | no relaxation | no relaxation | no relaxation | no relaxation |
| 10 | no relaxation | no relaxation | no relaxation | no relaxation |
| 12 | very slight | slight | no relaxation | no relaxation |
| | relaxation | relaxation | | |
| 14 | relaxation | relaxation | no relaxation | slight relaxation |
| 16 | relaxation | relaxation | slight relaxation | slight relaxation |
| 18 | relaxation | relaxation | slight relaxation | slight relaxation |
| 20 | — | — | some relaxation | slight relaxation |

The addition of ethoxydiglycol to the guanidine carbonate/calcium hydroxide reactive based systems did not enhance relaxation and/or straightening properties.

### Examples 4-7

Hair relaxer/straightener formulations were prepared having the compositions set forth in Table 4. The formulations were made by first preparing an oil phase comprising emulsifying wax (POLAWAX available from Croda, Inc., Parsippany, N.J., USA), mineral oil, petrolatum and cetearyl alcohol and a water phase comprising deionized water and, with the exception of Example 4, TRANSCUTOL CG. The ingredients of the oil phase were combined in a standard mixer and simultaneously mixed and heated to about 70° C. to about 75° C. In a separate standard mixer the ingredients of the water phase were combined and simultaneously mixed and heated to about 70° C. to about 75° C. (this step for Example 4 involved heating deionized water). The water phase was then added to the oil phase in the mixer and mixing continued while the contents were cooled to about 35° C. to about 40° C. to obtain an ethoxydiglycol phase. The ethoxydiglycol phase was allowed to sit at ambient temperature for about 24 hours after which a sodium hydroxide phase comprising sodium hydroxide and deionized water was added and mixed into the ethoxydiglycol phase at room temperature to obtain the hair care compositions.

Example 4 is a comparative example comprising no ethoxydiglycol and Examples 5, 6 and 7 comprise, respectively, 5%, 10% and 15% ethoxydiglycol, TRANSCUTOL CG, from Gattefossé. All formulations comprise 1.5% sodium hydroxide active.

About 4 grams of each relaxer/straightener composition was applied to separate hair tresses weighing about 1 gram each. The tresses were observed for up to 18 minutes or until relaxation and straightening was achieved, and the observations at various time increments after application are set forth in Table 5.

TABLE 4

| | Example 4 | | Exam- | |
|---|---|---|---|---|
| | (COMPARATIVE) | Example 5 | ple 6 | Example 7 |
| INGREDIENTS | wt/wt % | wt/wt % | wt/wt % | wt/wt % |
| Emulsifying | 10.0 | 10.0 | 10.0 | 10.0 |
| Wax NF | | | | |
| (POLAWAX) | | | | |
| Mineral Oil | 15.0 | 15.0 | 15.0 | 15.0 |
| (70 ssu) | | | | |
| Petrolatum | 20.0 | 20.0 | 20.0 | 20.0 |
| Cetearyl | 2.5 | 2.5 | 2.5 | 2.5 |
| Alcohol | | | | |
| Ethoxydiglycol | 0.0 | 5.0 | 10.0 | 15.0 |
| NaOH solution | 3.0 | 3.0 | 3.0 | 3.0 |
| Deionized water | 49.5 | 44.5 | 39.5 | 34.5 |

TABLE 5

| Time | Example 4 | | | |
|---|---|---|---|---|
| Min- | (COMPAR- | | | |
| utes | ATIVE) | Example 5 | Example 6 | Example 7 |
| 5 | no relaxation | no relaxation | no relaxation | no relaxation |
| 7 | no relaxation | no relaxation | no relaxation | no relaxation |
| 10 | no relaxation | no relaxation | slight relaxation | no relaxation |
| 12 | no relaxation | good | good relaxation | slight relaxation |
| | | relaxation | | |
| 14 | no relaxation | good | good relaxation | good relaxation |
| 16 | good | relaxation | | good relaxation |
| | relaxation | | | |
| 18 | good | | | |
| | relaxation | | | |

The relaxation time of 12 minutes (for the 5% (Example 5) and 10% (Example 6) ethoxydiglycol containing formulas) as opposed to 16 minutes for the comparative example without ethoxydiglycol is a reduction of about 25% in processing time.

About 2.5 grams of the formulations of Examples 4-7 as set forth in Table 4 were applied to separate tresses of hair weighing about 2.5 grams each. The tresses were observed for up to 16 minutes or until relaxation and straightening was achieved, and the observations are set forth in Table 6.

TABLE 6

| Time | Example 4 | | | |
|---|---|---|---|---|
| Min- | (COMPAR- | | | |
| utes | ATIVE) | Example 5 | Example 6 | Example 7 |
| 5 | no relaxation | no relaxation | no relaxation | no relaxation |
| 7 | no relaxation | no relaxation | no relaxation | no relaxation |
| 10 | some | some | some relaxation | no relaxation |
| | relaxation | relaxation | | |
| 12 | some | good | some relaxation | some relaxation |
| | relaxation | relaxation | | |
| 14 | good | good | good relaxation | some relaxation |
| | relaxation | relaxation | | |
| 16 | | | | good relaxation |

Relaxation for the 5% ethoxydiglycol containing product (Example 5) is 12 minutes compared to 14 minutes for the comparative example (Example 4) which represents a reduction in processing time of about 16%.

### Examples 8-10

Three sodium hydroxide based hair relaxer formulations were prepared in accordance with the procedures described for Examples 4-7 having the compositions set forth in Table 7. One comparative formulation had no ethoxydiglycol (Example 8), and the test formulations had 5% ethoxydiglycol (Example 9) and 10% ethoxydiglycol (Example 10), TRANSCUTOL CG from Gattefossé.

TABLE 7

| | Example 8 | | |
|---|---|---|---|
| INGREDIENTS | (Comparative) | Example 9 | Example 10 |
| Emulsifying Wax | 10.0 | 10.0 | 10.0 |
| NF | | | |
| Mineral Oil (70 ssu) | 15.0 | 15.0 | 15.0 |
| Petrolatum | 20.0 | 20.0 | 20.0 |
| Cetearyl Alcohol | 2.5 | 2.5 | 2.5 |
| Ethoxydiglycol | 0.0 | 5.0 | 10.0 |
| NaOH solution | 3.0 | 3.0 | 3.0 |
| Deionized water | 49.5 | 44.5 | 39.5 |

Each test formulation comprising the ethoxydiglycol (Examples 9 and 10) and the comparative example (Example 8) were subjected to salon testing using 10 subjects. A total of 20 subjects were evaluated, 10 subjects had their hair treated with the comparative formulation (Example 8) and the formulation of Example 9, and the other 10 subjects had their hair treated with the comparative formulation (Example 8) and the formulation of Example 10. The scalp of each subject was examined for cuts and abrasions and the hair was carefully combed to remove tangles and avoid scalp irritation. The hair of each subject was divided into four quadrants, from center part to back and from ear to ear over the crown. The control formulation was applied to two of the quadrants on the same side of the head and the respective example formulations were applied to the two quadrants on the other side of the head. Application for each product started at the nape of the neck quadrant and moved toward the front of the head. The products were applied to the root area of the hair, approximately ⅛ inch away from scalp and spread outward along the length of the hair strand. The cosmetologist used gloved fingers to distribute the test material. The formulations were applied quickly and evenly for complete coverage and uniform relaxation.

After application was complete, the cosmetologist smoothed the hair working from the back sections to front hairline, parting with one hand and smoothing with the other, with even strokes, using the flat sides of the fingers, not the tips. Extra test material was applied, if necessary, to ensure even coverage of hair from roots to end of a strand.

When exposure time for straightening was complete, the hair was rinsed until all the test material had been removed and the water ran clear. The hair was carefully examined to assure no test material remained in the hair and all product had been eliminated from the back of neck and hairline. The hair was shampooed thoroughly to neutralize the reaction. Conditioner was applied as needed. The results regarding the amount of time needed for hair treatment are set forth in Table 8.

TABLE 8

| | Application Time | | Smoothing Time | | Total Time | |
|---|---|---|---|---|---|---|
| Subj. No. | Example 8 (Comparative) | Example 9 | Example 8 (Comparative) | Example 9 | Example 8 (Comparative) | Example 9 |
| 1 | 2:50 | 2:30 | 1:38 | 0:43 | 4:18 | 3:13 |
| 2 | 2:48 | 2:00 | 1:13 | 1:04 | 4:01 | 3:04 |
| 3 | 2:21 | 2:30 | 1:30 | 1:20 | 3:51 | 3:50 |
| 4 | 2:51 | 3:07 | 2:18 | 3:00 | 5:09 | 6:07 |
| 5 | 2:46 | 2:04 | 1:37 | 1:19 | 4:23 | 3:23 |
| Total Time | 13:36 | 12:11 | 8:16 | 7:26 | 21:42 | 19:37 |
| Average | 2:43 | 2:26 | 1:38 | 1:29 | 4:20 | 3:55 |
| Subj. No. | Example 8 (Comparative) | Example 10 | Example 8 (Comparative) | Example 10 | Example 8 (Comparative) | Example 10 |
| 6 | 3:41 | 3:45 | 2:18 | 2:36 | 5:59 | 6:21 |
| 7 | 2:04 | 2:01 | 0:58 | 0:49 | 3:02 | 2:50 |
| 8 | 1:32 | 2:01 | 0:53 | 1:17 | 2:25 | 3:16 |
| 9 | 3:16 | 3:49 | 2:13 | 2:38 | 5:29 | 6:27 |
| 10 | 3:24 | 2:40 | 1:00 | 2:03 | 4:33 | 4:43 |
| Total Time | 13:57 | 14:16 | 7:31 | 9:23 | 21:28 | 23:39 |
| Average | 2:47 | 2:51 | 1:30 | 1:53 | 4:19 | 4:44 |

The salon testing indicates about a 9.6% reduction in overall processing time for the formulation containing 5% ethoxydiglycol. The subjects were questioned with respect to scalp reaction during application and smoothing time. There were no instances of stinging, itching, burning or irritation reported during or immediately after application.

The subjects returned four weeks after application of the control and test formulations. Observations of the subjects at that time revealed acceptable product performance. Also, none of the subjects reported scalp irritation.

1. A hair care composition for relaxing and straightening hair with reduced processing time for treating hair comprising from about 1% to about 10% ethoxydiglycol, sodium hydroxide, emulsifying wax, mineral oil, petrolatum, cetearyl alcohol and water.

2. The hair care composition of claim 1 comprising from about 0.5% to about 3% sodium hydroxide.

3. The hair care composition of claim 1 comprising from about 3% to about 6% ethoxydiglycol.

4. The hair care composition of claim 1 comprising about 5% ethoxydiglycol.

5. The hair care composition of claim 1 comprising about 1% to about 5% cetearyl alcohol, about 10% to about 35% petrolatum, about 5% to about 25% mineral oil, about 5% to about 20% emulsifying wax and about 30% to about 50% water.

6. The hair care composition of claim 5 comprising about 32% to about 45% water.

7. The hair care composition of claim 1 further comprising other active or inert ingredients selected from the group consisting of propylene glycol, glycerin, quaternium 80, panthenol, potassium cocoyl hydrolyzed collagen, PEG-60 lanolin, lanolin, steareth-20, laneth-15, PEG-150 stearate, dimethicone, dimethicone copolyol, cyclomethicone, ceteareth-20, PPG-5-ceteth-10 phosphate, polyquaternium-37, trideceth-7, PPG-1-trideceth-6, polysorbate 60, emulsifiers and combinations thereof.

8. The hair care composition of claim 1 further comprising colorings and/or fragrances.

9. The hair care composition of claim 1 wherein the water is deionized water.

10. A method for relaxing and/or straightening hair having reduced processing time comprising applying the hair care composition of claim 1 to human hair for a period of up to about 20 minutes and then rinsing completely.

11. The method of claim 10 wherein the hair care composition is applied in the form of a stiff cream.

12. A process for making a hair care composition for relaxing and/or straightening hair in reduced processing time comprising the steps of
a) forming an oil phase by combining cetearyl alcohol, petrolatum, mineral oil, emulsifying wax and water, and optionally other active or inert ingredients, colorings and/or fragrances, in a first means for mixing and simultaneously mixing and heating to a temperature of about 70° C. to about 75° C., and
b) forming a water phase by combining ethoxydiglycol and water in a second means for mixing and simultaneously mixing and heating to a temperature of about 70° C. to about 75° C., and
c) combining the oil phase and water phase in the first means for mixing, the second means for mixing or a third means for mixing and simultaneously mixing and cooling to a temperature of about 35° C. to about 40° C. to form an ethoxydiglycol phase, and
d) forming a sodium hydroxide phase by combining water and sodium hydroxide, and
e) combining and mixing the ethoxydiglycol phase and sodium hydroxide phase to obtain the hair care composition
wherein the hair care composition comprises about 1% to about 10% ethoxydiglycol.

13. The process of claim 12 wherein the hair composition further comprises about 0.5% to about 3% sodium hydroxide, about 1% to about 5% cetearyl alcohol, about 10% to about 35% petrolatum, about 5% to about 25% mineral oil, about 5% to about 20% emulsifying wax and about 30% to about 50% water.

14. The process of claim 12 comprising the additional steps of allowing the combined ethoxydiglycol phase and sodium hydroxide phase to stand at ambient conditions for up to about 24 hours after mixing then cooling the combined ethoxydiglycol phase and sodium hydroxide phase to about 35° C. to about 40° C. and homogenizing to obtain the hair care composition.

15. The process of claim 12 wherein the ethoxydiglycol phase is allowed to stand at ambient conditions for up to about 24 hours prior to combining the oil phase and water phase.

16. The process of claim 12 wherein the other active or inert ingredients are selected from the group consisting of propylene glycol, glycerin, quaternium 80, panthenol, potassium cocoyl hydrolyzed collagen, PEG-60 lanolin, lanolin, steareth-20, laneth-15, PEG-150 stearate, dimethicone, dimethicone copolyol, cyclomethicone, ceteareth-20, PPG-5-ceteth-10 phosphate, polyquaternium-37, trideceth-7, PPG-1-trideceth-6, polysorbate 60, emulsifiers and combinations thereof.

17. The process of claim 12 wherein the amount of ethoxydiglycol is 3% to about 6%.

18. The process of claim 12 wherein the amount of ethoxydiglycol is about 5%.

19. The process of claim 12 wherein the water is deionized water.
